(19) United States
(12) Patent Application Publication
(10) Pub. No.: US 2012/0264603 A1
(43) Pub. Date: Oct. 18, 2012

(54) ATTACHMENT AND RETENTION FORMULATIONS FOR BIOLOGICALLY ACTIVE ORGANIC COMPOUNDS

(76) Inventors: David S. Soane, Chestnut Hill, MA (US); Gangadhar Jogikalmath, Cambridge, MA (US)

(21) Appl. No.: 13/267,970

(22) Filed: Oct. 7, 2011

Related U.S. Application Data
(60) Provisional application No. 61/391,206, filed on Oct. 8, 2010.
(60) Provisional application No. 61/471,514, filed on Apr. 4, 2011.

(51) Int. Cl. C08G 63/91; A01N 39/04; A01N 37/10; A01N 43/40; A01N 37/22; A01N 47/24; A01N 35/10; A01N 25/10; A01N 25/34; A01N 43/707; A01N 43/50; A01N 47/34; A01N 43/78; C07C 59/70; C07C 59/68; C07C 65/21; C07D 213/83; C07C 233/18; C07C 311/53; C07C 251/52; A01N 25/26; A01P 13/00; A01P 7/04; A01P 3/00; C08G 65/48; A01N 39/02

(52) U.S. Cl. 504/101; 504/323; 504/324; 504/260; 504/342; 504/302; 504/344; 504/361; 504/359; 504/229; 504/253; 504/254; 504/329; 504/267; 514/772.5; 424/400; 562/472; 562/474; 546/315; 564/214; 560/12; 564/256; 525/419; 525/185

(57) ABSTRACT

The invention encompasses formulations for increased attachment and retention systems of biologically active organic compounds and methods for the preparation and use thereof.

### RELATED APPLICATIONS

This application claims the benefit of U.S. Provisional Application Ser. No. 61/391,206 filed Oct. 8, 2010 and U.S. Provisional Application Ser. No. 61/471,514 filed Apr. 4, 2011. The entire contents of the above applications are incorporated by reference herein.

### FIELD OF THE APPLICATION

This application relates to attaching and retaining biologically active ingredients to a variety of substrates for indoor and outdoor applications.

### BACKGROUND

Various biologically active ingredients are useful for agricultural and related purposes, for example pesticides, insecticides, anti-fungal agents and herbicides. These can exhibit a wide range of water solubilities, from insoluble/sparingly soluble, to moderately soluble, to highly soluble. Moderately soluble and highly soluble biologically active ingredients (AIs) are prone to loss due to erosion and leaching from treated soils and plants. Similarly, certain biologically active nutrients like water-soluble fertilizers that are applied to fields can suffer run-off or loss caused by rapid watering, rain or other water exposures. It is therefore desirable to develop a platform technology that allows for: 1) prolonged retention of AIs, 2) reliable, sustainable and tunable release kinetics, 3) minimum use of formulation materials, preferably at low cost, and 4) flexible applicability to a large array of AIs and nutrients.

As an example, the herbicides used in soil remediation are typically water soluble or sparingly soluble and their retention in the topsoil enhances the protection of crops. Of particular concern is the performance of -cidal agents that are pre-emergent, i.e., that are applied to the soil, for example, before the germination of plants and weeds, resulting in the suppression of weed growth. Pre-emergent agents need to stay where they are applied for a period of time while the plants and weeds are germinating. Dissipation of a pre-emergent agent by microbial activity, photodegradation, run-off by water exposure, and the like, needs to be minimized during the germination period, and it is important that the residence of the agent in the top one or two inches of soil is maintained during this period. While other -cidal agents face this same challenge, these problems are especially important for optimizing the behavior of systemic types of -cidal agents that affect the biological pathways in the undesirable organisms, as opposed to contact-type -cidal agents that kill immediately upon contact.

Herbicides are an example of an active ingredient category where improved retention is desirable after application. A number of these herbicides, in particular the water-soluble ones, suffer from poor retention in topsoil. Small herbicidal molecules used for anti-mold applications can also benefit from enhanced retention when they are applied. Furthermore, certain water-soluble compounds are known to photo-degrade. Both factors reduce herbicidal efficacy and durability. Therefore, a need exists to improve herbicide performance by: (1) enhanced retention of the active ingredients in the topsoil, (2) prevention of active ingredient leaching (i.e., sustained release), and (3) protection against photodegradation.

### SUMMARY

Disclosed herein, in embodiments, are attachment and retention systems for biologically active organic compounds and methods for the preparation and use thereof.

In one embodiment, the invention is directed to a formulation comprising: a polymer and a biologically active ingredient, wherein the polymer interacts with the biologically active ingredient by pi-pi stacking; and wherein the active ingredient is an aromatic compound. The invention also encompasses methods for the preparation of said formulation and methods for the use thereof. In certain embodiments, the biologically active ingredient is an agricultural active ingredient. The agricultural active ingredient can, for example, be an herbicide, an insecticide, and an anti-fungal agent. In certain aspects of the invention, the agricultural active ingredient is an herbicide. In certain additional aspects, the formulation comprises a styrene maleimide polymer or a styrene maleic anhydride polymer. In further aspects, the formulation comprises a polymer that possesses a chemical moiety capable of interacting with a substrate. Substrates include, for example, both non-agricultural and agricultural substrates.

The invention additionally encompasses a formulation comprising a biologically active ingredient and a particle, wherein the biologically active ingredient is directly or indirectly attached to a particle and wherein the biologically active ingredient is an aromatic compound that exerts a biological effect on a substrate. In additional embodiments, the invention encompasses methods for the preparation and methods for the use of the inventive formulation. In certain aspects, the active ingredient is an agricultural active ingredient. The agricultural active ingredient is, for example, an herbicide, an insecticide, and an anti-fungal agent. In certain aspects, the formulation comprises a particle which comprises a filler selected from the group consisting of precipitated calcium carbonate, clay, sand, diatomaceous earth, zeolite and silica. In certain additional aspects, the formulation comprises a polymer which is attached to the particle and wherein the polymer interacts with the active ingredient. The polymer can, for example, interact with the biologically active ingredient by pi-pi stacking.

In additional embodiments, the invention is directed to a formulation for sustained or controlled release of a biologically active ingredient comprising a core and a coating on the surface of the core, wherein the core comprises the biologically active ingredient and wherein the coating comprises a polymer and wherein the active ingredient is an aromatic compound that exerts a biological effect on a substrate. In some embodiments, the biologically active ingredient is an agricultural active ingredient. In certain additional embodiments, the agricultural active ingredient is selected from the group consisting of an herbicide, an insecticide, an anti-fungal agent, an insecticide and a fertilizer. In some embodiments, the polymer is a polycation.

The invention additionally encompasses a method of attaching or retaining an agricultural active ingredient on an agricultural substrate comprising: delivering to a treatment area a formulation described herein, wherein the agricultural active ingredient is an aromatic compound that exerts a biological effect on a substrate, and the treatment area comprises the substrate. The substrate includes, for example, soil, humus and/or a plant surface.

The invention further includes a method of controlling the release of a biologically active ingredient comprising delivering to a treatment area a formulation comprising a core and a coating on the surface of the core, wherein the core comprises the biologically active ingredient, the coating comprises a polymer and wherein the biologically active ingredient is an aromatic compound that possesses biological activity against a substrate; and the treatment area comprises the substrate.

### DETAILED DESCRIPTION

Disclosed herein, in embodiments, are attachment and retention systems for biologically active organic compounds and methods for the preparation and use thereof. In embodiments, certain organic compounds having biological activity can be used for agricultural purposes, for example as herbicides, anti-mold or antifungal agents, insecticides, and the like. These biologically active organic compounds, when used in an agricultural setting, can be termed “agricultural active ingredients.” These can be formulated advantageously for delivery to agricultural treatment areas where they can exert their biologically active properties when applied to agricultural substrates. The term “agricultural” is understood broadly herein, to include commercial agriculture, residential and institutional lawn and garden uses, and the like. In other embodiments, biologically active organic compounds having pesticidal, anti-fungal or anti-mold properties can be applied to non-agricultural substrates such as building materials, carpeting, wall coverings, and other treatment areas where a durable retention of such a biologically active ingredient would be advantageous. These biologically active organic compounds applicable to non-agricultural substrates may be chemically similar to those biologically active organic compounds used in agricultural settings as pesticides, antifungal or antimold agents, and the like, or they may be chemically different than those having specific agricultural applicability.

As used herein, the term “treatment area” refers to any area where the effect of the small molecule active ingredient in question would otherwise be desirable, e.g., as a herbicide, anti-mold or antifungal agent, or as an insecticide. As used herein, the term “active ingredient” refers a biologically active organic compound comprising carbon atoms and optionally including heteroatoms such as boron, nitrogen, oxygen, phosphorus, sulfur or selenium, formulated as a small molecule or as a composite payload comprising organic molecules of different sizes, such as polypeptides, protein fragments, small molecules, oligomers and macromolecules, where the biologically active organic compound can be delivered to a designated treatment area to have a desired pharmacological effect, i.e., herbicidal, insecticidal, fungicidal, nutrient delivery, etc. Active ingredients suitable for treatment in accordance with these systems and methods can include water-soluble active ingredients and sparingly soluble active ingredients. Examples of water-soluble active ingredients include herbicidal and pesticidal agents such as a triazine or triazinone (e.g., the triazines atrazine and cyanazine, and the triazinone metribuzin), an alachlor (chlorinated acetamide), a benazolin, a bentazone, an imazapyr or triclopyr, or a sulfonyl urea. Examples of sparingly soluble active ingredients include herbicidal and pesticidal agents such as metolachlor and various sulfentrazones (e.g., butsulfentrazone). The target for the active ingredient is the substrate or surface upon which its attachment is desirable so that the active ingredient can exert its desired effect (e.g., curtailing unwanted weed growth, insect pests, mold, and the like, enhancing crop growth, etc.). Exemplary targets include, for example, soil, plant surfaces and humus.

In embodiments, attachment and retention systems can be created using multi-block copolymers or graft copolymers as specially designed carriers for the small molecule compounds of interest, where one moiety of the copolymer interacts with the small molecule active ingredient strongly while another moiety of the copolymer attaches to soil (sand/clay) or other target. In embodiments, the copolymer carrier acts as a binder to attach the active ingredient to the target. The attachment and retention properties of these binder-based formulations can be tuned so that they provide for a precisely engineered effect of time-determined efficacy when the active ingredient encounters the target.

In other embodiments, particle-based formulations can provide for sustained or controlled release of one or more active ingredients, by associating the active ingredients with a variety of particles. Particles carrying the active ingredients can then be dispersed across treatment areas, using delivery techniques familiar to skilled artisans. The attachment and retention properties of these particle-based formulations can be tuned so that they provide for a precisely engineered effect of sustained or controlled release.

As an example, attachment polymers can be used to attach the active ingredients to a variety of particles. In certain embodiments, the particle surface can be functionalized by pretreating it with polymers that afford points of attachment binder polymers as described above that have affinity for the active ingredients as well as affinity for a target substrate. In other embodiments, functionalization is not necessary, and binder polymers can be used to attach the active ingredients directly to the non-functionalized particles. In yet other embodiments, porous particles can be used as carriers for the active ingredients, where the active ingredients are imbibed into the porous particles, optionally with the use of a binder polymer to attach the active ingredient therein and/or to attach the porous particle to a target substrate.

In other embodiments, formulations for sustained or controlled release of one or more active ingredients can be formed as composites comprising a central core having a surface encapsulation or “skin.” In embodiments, the core can comprise the active ingredient that is deployed as discrete deposits within a matrix or comingled with the matrix itself, in arrangements designed to control the delivery of the active ingredient. In embodiments, the surface coating or encapsulation can comprise one or more polymers having affinity for the surfaces of a target, and the encapsulation can be formulated of any advantageous thickness, from a monolayer or partial monolayer to a thicker coating with multiple layers of one or of several different polymers. The encapsulation may be formed homogeneously, or it may comprise different polymers in different regions or in different layers, each imparting specific properties.

In embodiments, a polymer-small-molecule system designed in accordance with these attachment and retention mechanisms (i.e., a binder-based formulation, a particle-based formulation, or an encapsulated-core formulation) can be delivered to the treatment area using conventional delivery methods, so that the active ingredient attains its goal of controlled action upon the target, for example, the goal of destroying or curtailing the growth of the target in question, destroying pests, delivering a nutrient, and the like. In embodiments, use of a polymer-small-molecule system as described herein permits enhanced retention for the active ingredients in the treatment area and prevents their leaching therefrom.

In certain embodiments, systems formulated in accordance with these systems and methods offer the further advantage of diminished photo-degradation for the active ingredients. In the binder-based formulations system, for example, binders designed with affinity for the aromatic composition of certain active ingredients can include copolymers that are primarily aromatic in structure, where this aromaticity can impart ultraviolet opacity to the resulting complexes. In the particle-based system, for example, porous particles can be selected for containing the active ingredients that are intrinsically opaque to ultraviolet wavelengths, due to light scattering accompanying the porous morphology of such “containers.” In the encapsulated-core system, encapsulation polymers can be selected to dissipate ultraviolet radiation, or encapsulation polymers can be formulated that incorporate nano- or micro-particles that reflect/attenuate/dissipate ultraviolet radiation. In embodiments, for example, binder or encapsulation polymers such as SMI or the LCST polymers can be mixed with polymers containing conjugated structures such as double bonds or with a small amount of carbon black. These compounds absorb UV light and shield the active ingredient compounds which might be photodegradable.

### 1. Binder-Based Formulations

In embodiments, a binder-based attachment and retention system can be designed for active ingredient compounds that have an aromatic structure, where the binders comprise polymers containing aromatic structures that interact with the active ingredient(s) via pi-pi stacking interactions. Not to be bound by theory, a chemical compound with phenyl rings, for example, can be associated with an aromatic-containing polymeric matrix by the use of pi-pi stacking involving flat aromatic structures with pi electron clouds that overlap with neighboring aromatic structures resulting in strong interactions between them. For example, phenoxy acid herbicides (e.g., 2,4-D (WEEDONE®), mecoprop (MCPP) and 2,4-D plus 2,4-DP (WEEDONE® DPC)), benzoic acid herbicides (e.g., dicamba) and pyridine herbicides (e.g., dithiopyr), arylaniline herbicides (e.g., benxyloprop), chloroacetanilide herbicedes (e.g., metalochlor), sulfonamide herbicedes (e.g., asulam), phenoxy herbicides (e.g., bromofenoxim) are characterized by an aromatic structure and can demonstrate pi-pi stacking association with polymers such as those disclosed herein. To create a polymeric binder-based attachment and retention system, polymeric additives having aromatic structures, for example, can be selected so that they are affixable to or otherwise associable with various targets, for example, components of the soil for outdoor use, or surfaces of building materials such as facing paper of wallboard for indoor use, so that the polymeric additive bearing the aromatic active ingredient would be attracted thereto.

As used herein, the term “aromatic” includes entitles having aromatic rings such as 5-, 6-, and 7-membered single-ring aromatic groups that may include from zero to four heteroatoms. Examples include chemical compounds possessing benzene (benzyl), pyrrole (pyrrolyl), furan (furanyl), thiophene (thienyl), imidazole (imidazolyl), oxazolo (oxazolyl), thiazole (thiazolyl), triazole (triazolyl), pyrazole (pyrazolyl), pyridine (pyridinyl), pyrazine (pyrazinyl), pyridazine (pyridazinyl) and pyrimidine (pyrimidyl) groups, and the like. Those aromatic groups having heteroatoms in the ring structure can also be referred to as “aryl heterocycles” or “heteroaromatics.” Heteroatoms are atoms other than carbon or hydrogen. In some instances, heteroatoms can be any one of boron, nitrogen, oxygen, phosphorus, sulfur and selenium.

The aromatic ring can be substituted at one or more ring positions with substituents, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aromatic” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. The term “aromatic” also includes polycyclic rings systems having two or more cyclic rings which are covalently attached, wherein at least one of the rings is aromatic.

In embodiments, a styrene maleimide (“SMI”) polymer can be employed as a binder in a binder-based attachment and retention system. For example, polymers made with styrene and maleimide monomers (i.e., SMI polymers) can be solubilized in acidic aqueous solutions and can possess cationic charges. The cationic groups of a SMI polymer can bond electrostatically to negatively charged components of soil such as sand and clay. As another example, a SMI polymer can be precipitated onto a substrate such as an anionic particle by increasing the pH of the solution. This is a reversible process, and the SMI can also be re-solubilized by again lowering the pH to a sufficient level. SMI polymers can concomitantly retain aromatic active ingredients by pi-pi stacking, so that such active ingredients can be delivered to the delivery area and be retained there.

It would be understood by those of ordinary skill that other polymers having phenyl groups or other aromatic configurations can be used similarly to create an aromatic surface for compounds with phenyl rings to bind to. In embodiments, for example, other configurations of SMI polymers can be used for these applications, e.g., where the styrene to maleimide ratio is varied, creating either more phenyl groups or maleimide groups on the surface of the particles. The additional phenyl groups on the polymer can cause the polymeric substrate to exhibit increased hydrophobicity. Not to be bound by theory, it is understood that increased hydrophobicity can limit access to the payload, thereby limiting its release over time.

### 2. Particle-Based Formulations

In certain embodiments, attachment polymers can be associated with various particulate materials, such as filler materials, to provide attachment points for highly aromatic active ingredients so that the active ingredient is affixed to or closely associated with the surface of the particulate. Fillers can include materials such as precipitated calcium carbonate (“PCC”), clay, sand, diatom, zeolite, porous silica, and the like. As an example, a polymeric “binder” like SMI can be precipitated onto a filler like PCC or silica. Such a binder can also interact with an aromatic active ingredient, for example by pi-pi stacking, to attach the active ingredient to the particle. The polymeric binders for attaching the active ingredients onto the fillers can have affinity for a number of active ingredients, such as those containing aromatic moieties as previously discussed, allowing the active ingredient(s) to be affixed to or closely associated with the surface of the filler. Using a filler or other particle as the attachment vehicle for the active ingredient(s) can facilitate their dispersal on targets and make them harder to dislodge when the targets encounter rain or flood waters.

In embodiments, a variety of polymers containing phenyl groups can be used to form retention systems for aromatic compounds. As an example, styrene maleic anhydride (“SMA”) polymers can be used for these purposes, either in a binder-based retention system or in a particle-based system. For use with particles, though, an extra step needs to be taken, because the SMA polymer does not show pH-mediated precipitation onto surfaces. Instead, the surface of the particle can be modified with another agent that permits the attachment of the SMA thereto. For example, chitosan can be applied to the particle as an overcoat, either by pH-mediated precipitation or by electrostatic attraction on a charged surface such as that of PCC. Chitosan acts as a surface modifier for the particle that attaches to the SMA. By coating the particle with a polyamine-containing attachment agent such as chitosan, the amine groups of the polyamine-containing binding agent layer (e.g., the chitosan layer) can react with the anhydride groups of the SMA to associate the SMA with the particulate material. The particles bearing the SMA can then be associated with one or more designated aromatic active ingredient(s) in a manner similar the method described above for associating active ingredients with particles bearing SMI.

In other embodiments, two different formulations can be prepared as described above, for example, a SMA-based formulation bearing one active ingredient, and a SMI-based formulation bearing another active ingredient. The two formulations can then be combined within a single system, yielding dual activity and/or controlled dosage. In certain embodiments, a SMA-based formulation can be provided to contribute reactive groups for binding an active ingredient, and a SMI-based formulation can be provided to contribute cationic groups to bind the soil. If one or more of the active ingredients are bound to one or more particles, the combination of formulations can yield a single, composite particle system.

In other embodiments, copolymers of polyethylene glycol (“PEG”) or polypropylene glycol (“PPG”) and polystyrene can be used for associating aromatic active ingredients with particles. When such a polymer is used, the PEG or PPG components of the polymer can allow it to be precipitated onto the filler by increasing the temperature above the LCST (lower critical solution temperature) of the polymer. Raising the temperature above the LCST can render the polymer insoluble, thereby permitting it to coat the substrate.

In this configuration, the polystyrene component of the polymer, for example, can permit association with aromatic compounds that are active ingredients via pi-pi stacking, while the PEG or PPG component permits association of the polymer with the substrate, e.g., with particles and/or with the soil or other substrates. For example, a mixture of a “cidal” active ingredient (for example, herbicidal, pesticidal, and fungicidal active ingredients) with an LCST-hydrophobic copolymer can be directly sprayed on a substrate such as soil with the LCST block precipitating onto the soil particles. Or the active ingredient can be introduced into a porous particle system with a LCST polymer acting as an overcoat controlling the release of the active ingredient. In embodiments, other copolymers that combine components exhibiting an LCST with components containing a phenyl group would be suitable for these applications.

In embodiments, retention systems can be created using small porous particles, which can be natural, synthetic, organic, or inorganic, to imbibe the small molecule compounds of interest into small porous particles, so that active-ingredient-bearing “packets” can be prepared having suitable dimensions for delivery to the treatment area. For example, small vesicular structures can be used ranging in size from tens of nanometers to hundreds of microns. These small “packets” containing the active ingredient are then surface-coated with an ultrathin polymer layer that serves two functions: prolonged release (with controlled leaching kinetics) and high affinity towards soil components. The ultrathin polymer layer may be further augmented with water dispersible molecular branches such as PEG, PEO, carbohydrates, sugar molecules (e.g., dextrans), and the like, to promote colloidal dispersion stability, so the formulation remains shelf-stable rheologically and can be easily sprayed or applied to the treatment area.

In embodiments, a system for delivering active ingredients in a controlled manner into the environment and for ensuring their durability in the treatment area can comprise porous particles as a retention system. Using such a system, active ingredients (either water soluble or sparingly soluble) can be incorporated into small “packets” (micron size or larger) made from specifically engineered particulate matter comprising such materials as starch granules, polyacrylic acid (various crosslinking degrees and sizes), zeolite, diatomaceous earth, or porous silica. Once fully imbibed in the porous structure, the loaded packets bearing the active ingredient can be over-coated with an ultrathin (i.e., monolayer or bi-layer) self-assembling polymer that controls release kinetics and provides simultaneous soil adherence.

In one embodiment, a water-soluble active ingredient, e.g., an herbicide compound, can be mixed with a 1% starch solution. This starch solution can then be crosslinked with the use of crosslinkers such as glyoxal or glutaraldehyde into a solid mass. The solid mass can then be crushed to make smaller particles that are porous and that have the active ingredient loaded inside.

In another embodiment, a water insoluble active ingredient, e.g., an aromatic herbicide (e.g., with phenyl rings), can be mixed with a block copolymer of SMI or SMA so as to associate the active ingredient with the polymer. This system can then be mixed with an anionic particulate carrier such as zeolite, diatomaceous earth or porous silica. The SMI would bind to the particle via electrostatic attraction. The carrier system can then be coated with a thin layer of cationic polymer such as chitosan, polyvinylamine, or the like. The external layer of cationic polymer acts as to attach the particle-based formulation to targets, for example soil components such as sand or clay.

In another embodiment, the porous carrier particles that are loaded with active ingredient(s) can be coated with a thin layer of another polymer such as carboxymethylcellulose or dextran or other similar biodegradable polymers before being coated with a cationic polymer. The presence of biodegrading polymer enables the slow release of the active ingredient from the interior of the particle as the coating is degraded or, e.g., consumed slowly by bacteria that reside in the delivery area.

### 3. Encapsulated Core Formulations

a. General Principles

In certain embodiments, formulations for sustained or controlled release of one or more active ingredients can be created as composites where a core comprising an active ingredient is associated with, enveloped by, or otherwise coated with a selected polymer (such processes all being considered examples of “encapsulation”). In embodiments, encapsulated core formulations can be formed as composites comprising a central core particle having a surface coating or “skin.” In embodiments, the core can comprise an active ingredient that is surrounded by or comingled with a small amount of an engineered matrix that controls the release and delivery of the active ingredient. In embodiments, the surface coating or encapsulation can comprise one or more polymers having affinity for the formulation's target substrates (e.g., soil or plant surfaces).

In embodiments, LCST polymers can be used to encapsulate the active ingredients. For example, a gel made of crosslinked PPO-PEO copolymer with an LCST of 20° C. can be used. The active ingredient can be mixed with prepolymer at temperature below LCST. Then the crosslinking reaction is initiated either by providing a crosslinking agent or by adding another polymer that will crosslink but will form an interpenetrated network with the LCST polymer when the temperature of the polymer solution is raised above LCST (resulting in precipitation of LCST polymer). Thus encapsulated, the active ingredient can be further encased in a cationic polymer, such as chitosan, which can be precipitated onto the gel core containing the active ingredient, enabling the encapsulated system to attach to soil or other substrates. In embodiments, an encapsulation such as that provided by a LCST polymer can provide for enhanced and tunable sustained release, and can protect the active ingredient against breakdown.

In other embodiments copolymers of acrylic monomers can be designed and used to create encapsulations for the carrier systems that contain active ingredients, e.g., herbicides. These acrylic compounds are temperature sensitive, with their Tg (glass transition temperatures) being tuned to soften when the temperature of the soil surrounding them reaches appropriate temperature. With softening, the encapsulation allows controlled release of the active ingredient.

In accordance with these systems and methods, cores can be formulated that provide for slow release and controlled water permeation, for example by possessing nanochannels or sparing intrinsic water solubility, so that the discharge of the active ingredient into the environment is prolonged. For these uses, a core element can contain a mass of the active ingredient that is coated as a unit, or it can contain a plurality of active ingredient deposits dispersed throughout a continuous matrix, with each deposit optionally coated with a specific coating agent that affects its release kinetics. By providing certain deposits of an active ingredient with a specific coating within the matrix, the release characteristics for the active ingredient can be further engineered in accordance with a predetermined design. For example, with two different active ingredients, individualized coatings around each within the matrix can allow one to be released faster than the other based on the nature and/or the thickness of the coating.

In an exemplary embodiment, the encapsulated core formulation can be constructed as a core element surrounded by an encapsulation or a “skin.” In embodiments, the core can be encased in an ultrathin outer layer “skin,” or encapsulation which can be a monolayer or near-monolayer of a polycation. In other embodiments, the encapsulation can comprise a plurality of layers formed from a single polymer or from multiple polymers, arranged for example in a patchwork, network, or onion-skin configuration.

Composite encapsulated core formulations as described herein can provide a precision-engineered delivery system for a concentrated active ingredient, allowing the active ingredient residing on the target to be protected against rapid loss. The encapsulated core formulation particles can simultaneously exhibit sustained release of the active ingredients, controllably tunable kinetics, high loading capacity, high affinity binding to substrates, and environmentally-friendly yet low-cost “packaging” matrices. Exemplary active ingredients that can be formulated as encapsulated cores can include a variety of biologically active compounds for agricultural uses (herbicides, fungicides, insecticides, fertilizers, and the like) such as those comprising triazines (atrazine and cyanazine), alachlor (chlorinated acetamide), benazolin, bentazone, imazapyr and triclopyr, sulfonyl urea based herbicides, and the like.

b. Encapsulation Polymers

In embodiments, polycations are useful polymers for the encapsulation layer in encapsulated core formulations. Desirable polycations can be selected according to their hydrophilicity and cationic charge density. Because the charges on the target substrates, e.g., soil, are largely anionic in nature, these characteristics for encapsulation polymers can enhance their attraction to targets. In embodiments, the encapsulation polymers can be naturally derived, for example, proteins and glucosamines. In embodiments, the encapsulation polymers can have a switchable solubility profile, as a function, for example, of pH, temperature or ionic strength, to enable the facile deposition of these encapsulations on the core matrices bearing the active ingredient(s). Exemplary polycations include cationic proteins or glycoproteins, SMI (imidized styrene maleic anhydride), zein, casein, or any of a number of polyamines (such as polyvinylamine, polyallylamine, polyethylenimine), and their derivatives (such as PEGylated varieties), DADMAC, chitosan (and its derivatives including different degrees of hydrolysis from chitin), and the like. Depending on the selected encapsulation polymer, application techniques can be selected that would be familiar to those having ordinary skill in the art. As an example, in embodiments polymers can be controllably deposited on the core matrix by precisely titrating the pH of the system in certain embodiments. As another example, in embodiments the encapsulation can be applied to the core by heating it to an elevated temperature, similar to the process used for sugar coating confectionaries. In yet other exemplary embodiments, the encapsulation can be applied using a high-shear device, where intense shearing provides an even and thin encapsulation around the core. In additional embodiments, the active ingredient may be formed into a plurality of kernels, each one of which is to be coated with the encapsulation layer, with the coated kernels to be assembled within a core matrix. In such embodiments, a high-shear device can be used to apply an encapsulation layer over the plurality of kernels.

c. Core Matrices

Core matrices are intended to retain the active ingredient(s) and allow for such active ingredient(s) to be released in a controlled manner after the formulation has contacted the target. A number of processes are available for forming a core matrix that holds the active ingredient(s). Core matrices, once formed, are encapsulated with a polymeric encapsulation layer, as described previously.

In embodiments, the inner core can be formed through an anhydrous process, beginning with one or more solid active ingredient powders, and then adding up to four other substances form the core matrix: wax, oil, olefin polymer or co-polymer and fatty acid. Wax is a useful and versatile component for forming the core matrix. Wax is paraffin and has a low melting temperature; suitable waxes include materials like candle wax and beeswax, or any other low melting solid paraffin that is approved for soil and/or agricultural use. Under high shear conditions, wax melts so that it is sufficiently flowable to be combined with the active ingredients. Wax is hydrophobic, thus protecting the inner core from water ingression and facilitating the sustained release of the active ingredient.

In the absence of wax, or in addition to wax, the inner core matrix can comprise one or more olefin polymers or oils. Oil can be added to the matrix formulation to tune further the water ingression rate into the matrix over orders of magnitude. Oils are selected that are compatible with the wax. In case higher water penetration rates are desired, a small amount of glycerin can be co-added with the oil. Suitable oils include vegetable oils (e.g., palm oil) to maximize the “bio-derived” characteristic of the formulation, or mineral oils. A small amount of an olefin polymer or co-polymer can be added to the matrix to preserve the geometrical integrity of the final encapsulated product, especially advantageous, for example, in hot climates. Furthermore, a fatty acid, such as stearic acid, can be added to impart additional water compatibility and ensure a negative surface charge on the core so that adherence of a polycation encapsulation is facilitated. The hydrophobic tail of the fatty acid makes it compatible with the wax base, while its hydrophilic head attracts the polycation encapsulation.

In accordance with certain embodiments, the various matrix ingredients can be first mixed thoroughly in a high shear mixer, and then broken into fine particles. In embodiments, a non-polar but volatile solvent can be used to assist mixing. The resulting matrix mixture can then be added to a high shear mixer containing the active ingredient at a matrix:active ingredient ratio of less than 1:1 to minimize consumption of the matrix ingredients.

In other embodiments, the inner core can be formed through an anhydrous process using a dilute polymer solution in a non-aqueous solvent. In embodiments, the matrix material can be a derivatized cellulose, for example, acetylated, propylated, butyrated cellulose (single and multiple substitutions) and poly-ethers of derivatized celluloses, and copolymers or mixtures of these two groups. This sort of matrix material can be combined with one or more volatile and benign solvents such as acetone and/or isopropyl alcohol as a casting solvent. To incorporate the active ingredient(s) into the matrix material, a free-flowing powder of active ingredient(s) is added slowly to a dilute solution of the matrix material in a mixing vessel. When the solvent is evaporated and the mixture is agitated, an even, thin coating of matrix material is deposited on the active ingredient(s). Plasticizers such as glycerin and PEG can be added into the matrix material to tune the release kinetics of the final active-ingredient-containing particles.

In other embodiments, an inner core can be formed through a process that uses a small amount of water. As the initial component, a highly absorbent material such as crosslinked PAA (i.e., polyacrylic acid, which exists in the salt form at neutral pH) is selected, which is capable of imbibing great quantities of a concentrated aqueous solution. This material can be formed as particles or beads that are initially dry. Then, one or more water-soluble active ingredient(s) can be dissolved in a small amount of water to make a highly concentrated solution. When the dry absorbent powder is mixed with the concentrated solution of active ingredient(s), the mixture quickly turns into a thick paste, with a large proportion of the active ingredient becoming incorporated within the adsorbent particles or beads. At this point, little residual aqueous solution is left in the interstitial space between the swollen particles or beads of the absorbent.

After incorporating the active ingredient into the absorbent particles or beads, a cationic polymer can be added to the matrix, with the polymer precipitating upon the particle or bead surface. Strong charge-charge attraction leads to rapid polymer deposition, which immediately seals the active ingredient(s) within the beads. Previously imbibed water within the particles or beads can still escape, and this evaporation can be enhanced with the application of heat. Sufficient evaporation yields a dry flowing powder of absorbent material (e.g., PAA) that contains the incorporated active ingredient(s), with a thin surface coating of the cationic polymer. This method of preparation produces a matrix that controls active ingredient release by the strong bi-polymer interaction of the cationic topcoat and the anionic crosslinked matrix. To further control and/or restrict release of the active ingredient(s), the polycations used for the surface encapsulation may be derivatized with hydrophobic side groups. For example, chitosan or polyamines may be pre-reacted (or derivatized post-deposition) with short-chain aliphatics with an epoxy group.

In some embodiments, the invention is directed to formulations for delivering an active ingredient to a substrate, comprising a core matrix containing an encapsulated ingredient, and a polymeric coating disposed upon the core matrix, wherein the polymeric coating has an affinity for the substrate. In embodiments, the core matrix is anhydrous. In other embodiments, the core matrix comprises a water-absorbent material. In embodiments, the polymeric coating comprises a polycation. In embodiments, the active ingredient can be selected from the group consisting of triazines (atrazine and cyanazine), alachlor (chlorinated acetamide), benazolin, bentazone, imazapyr and triclopyr, and sulfonyl urea based herbicides.

### EXAMPLES

### Materials

SMA® 1000P, Sartomer, Exton, Pa.
SMA® 1000I, Sartomer, Exton, Pa.
SMA® 2000I, Sartomer, Exton, Pa.
SMA® 3000I, Sartomer, Exton, Pa.
Chitosan CG10, Primex, Siglufjordur, Iceland
Chitosan CG110, Primex, Siglufjordur, Iceland
ViCALity ALBAGLOS USP/FCC Precipitated Calcium Carbonate, Specialty Minerals, Bethlehem, Pa.
Silica, fumed, 7 nm, Sigma Aldrich, St. Louis, Mo.
Hydrochloric Acid, ACS reagent, Sigma Aldrich, St. Louis, Mo.
Sodium Hydroxide Pellets, ACS reagent, Electron Microscopy Science, Hatfield, Pa.
Glycerin
Stearic acid
Zein, Freeman Industries
Poly(ethylene-co-vinylacetate)
Crosslinked Poly(acrylic acid) beads, Aldrich
Sulfentrazone granules
Metribuzin granulated powder

### Example 1

### Water Solubility of Styrene Maleimide (“SMI”)

Styrene maleimide (“SMI”), at three different ratios of styrene to maleimide (SMA® 1000I, SMA® 2000I, and SMA® 3000I), was added to water with amounts of 1M HCl to solubilize it. A pH of 4-4.5 was used to create an aqueous solution of SMA® 1000I, SMA® 2000I, and SMA® 3000I. These results are consistent with the statements in Sartomer Application Bulletin 4957 “SMA® Imide Resins SMA® 1000I, 2000I, 3000I, and 4000I”, that a pH of 4.5 is required for solubilizing the polymers. Each aqueous solution was then titrated using a base such as NaOH until the polymer precipitated out of the solution, typically at a pH of about 8. Adding acid again to reduce the pH once again solubilized the SMI.

### Example 2

### Preparation of Chitosan Solution

A chitosan solution of CG10 was prepared by dispersing CG10 in deionized water and adding 1M HCl until the chitosan was dissolved. The final pH was approximately 3.5. Chitosan solutions were then further diluted with deionized water to obtain the concentrations set forth in the Examples below.

### Example 3

### Zein Modification of Substrates

A 0.1% solution of Zein was made by diluting 14% Aquazein (Freeman Industries) in basic water (˜10 pH). A 50 g sample of sulfentrazone in granulated form was suspended in a 1 liter solution of 0.1% Zein, and pH was lowered to ˜5 using dilute HCl, while stirring to enable Zein deposition on the substrate. The solution was then drained and the modified substrate dried overnight at 25° C.

### Example 4

### Wax Encapsulation of Water Soluble AI (Active Ingredient)

1 gm of wax (Aldrich, m.p 55 C) and 9 g of metribuzin were dry-mixed in a plastic container. The container was then loaded into a high shear mixer (FlackTek DAC 150 FVZ-K (FlackTek, Landrum, S.C.)). The mixture was shear mixed at ˜2000 rpm for 10 mins. The high shear melted the wax, thereby coating the AI granules. The thickness of the encapsulation material was varied by altering the wax:AI stoichiometry.

### Example 5

### Retention Studies of SMI onto Particles

In this Example, SMI was adsorbed onto different particle surfaces by controlling the pH. 10 g of silica particles were suspended under agitation in 1% solution of SMA® 1000I solution in pH 4-4.5. The pH was then raised to precipitate the SMA® 1000I onto silica particles according to the methods set forth in Example 1. Retention of the SMA® 1000I was correlated with the measured hydrophobicity of the samples: where hydrophobicity is higher, the retention was better. The experiment was repeated with PCC and cellulose fibers as particle substrates.

### Example 6

### Retention of Herbicides by pi-pi Stacking

Herbicides of the phenoxy class and aromatic acid classes and others such as sulfentrazone having aromatic rings can be retained in aqueous solutions that contain SMI copolymers. For this Example, 10 g of sulfentrazone, which is soluble in water at 7.5 pH, was mixed with a 1% solution of SMA® 1000I at pH 5 such that the ratio of sulfentrazone to dry weight of SMA® 1000I in the solution was 99 to 1. As the pH of the solution was raised, the solubility of sulfentrazone increased while solubility of SMA® 1000I was reduced, improving the association of herbicide with the aromatic rings of SMA® 1000I. At around pH 8, SMA® 1000I precipitated onto the herbicide, encapsulating it. This solution was then used to spray onto soil.

### Example 7

### Encapsulation of Herbicides by Porous Silica

A 1% solution of sulfentrazone at 7.5 pH in water can be mixed with 5 g of porous silica particles suspended in DI water. The pH of the solution is slowly reduced to ˜5 where sulfentrazone is sparingly soluble in water. This facilitates the precipitation and binding of the sulfentrazone molecules to the walls of the porous silica. The water is drained and the porous silica is then resuspended in a 1% solution of chitosan at pH 3.5 that is prepared as described in Example 2. The pH of the suspension is raised to ˜7 to enable precipitation of the chitosan on the surface of the porous silica. This layer of chitosan acts to attach the porous silica particles to soil and can provide a slow release barrier for sulfentrazone molecules.

### Example 8

### Retention of Herbicide Molecules in Crosslinked Starch Particles

A 1% solution of cationic starch in water (e.g., Stalok 365) can be mixed with a predetermined quantity of sulfentrazone in water at 7.5 pH. The mixture is vortexed and treated with a 1% solution of glyoxal. The mixture is then vortexed and dried to obtain a crosslinked mass of starch with sulfentrazone trapped inside. The starch mass is then crushed using a ball mill to obtain uniform sized crosslinked granules containing sulfentrazone which can then be applied to the soil. Suitable hydrophobically modified starches such as FilmKote54 or FilmKote550 can be used in place of cationic starch or mixed with cationic starch to enable retention of hydrophobic herbicides.

### Example 9

### Hydrophobic Encapsulation of AI with Wax with an Oil Diluent

To enhance the biodegradability of the formulation that encapsulates the AI, bioderived oils such as vegetable oils (corn, peanut, etc.) can be added to the encapsulation formulation. For this Example, 0.7 g of wax (Aldrich, m.p 55 C) and 0.3 g of vegetable oil were dry mixed in a plastic container. To this container, 9 g of metribuzin was added. The container was then loaded into a high shear mixer (FlackTek DAC 150 FVZ-K (FlackTek, Landrum, S.C.)). The mixture was shear mixed at ˜2000 rpm for 10 mins. The high shear melted the wax and formed an encapsulation on the metribuzin granules. The thickness of the encapsulation material can be varied by altering the wax:metribuzin stoichiometry. Changing the stoichiometry of oil in the encapsulation composition can alter the release kinetics of the encapsulated material.

### Example 10

### Hydrophobic Encapsulation with Wax and with a Fatty Acid Diluent

To enhance the biodegradability of the formulation that encapsulates metribuzin, bioderived fatty acids such as stearic acid can be added to the formulation. For this Example, 0.7 g of wax (Aldrich, m.p 55 C) and 0.3 g of Stearic acid (Aldrich) were dry-mixed in a plastic container. To this container, 9 g of metribuzin was added. The container was then loaded into a high shear mixer (FlackTek DAC 150 FVZ-K (FlackTek, Landrum, S.C.)). The mixture was shear mixed at ˜2000 rpm for 10 mins. The high shear melted the wax and stearic acid and formed an encapsulation on the AI granules. The thickness of the encapsulation material can be varied by altering the wax:AI stoichiometry. The release kinetics of the encapsulated material can be altered by changing the stoichiometry of fatty acid in the formulation. The anionic group on the stearic acid molecule provides an attachment point for cationic polymers that are useful for soil binding.

### Example 11

### Hydrophobic Encapsulation with a Glycerin Diluent

To enhance the biodegradability of the formulation that encapsulates the AI, glycerin can be added to the formulation. 0.7 g of wax (Aldrich, m.p 55 C) and 0.3 g of glycerin (Aldrich) can be dry-mixed in a plastic container. To this container, 9 g of an AI can be added. The container is then loaded into a high shear mixer such as the FlackTek DAC 150 FVZ-K (FlackTek, Landrum, S.C.). The mixture is shear mixed at ˜2000 rpm for 10 mins. The high shear melts the wax and stearic acid and forms a coat on the AI granules. The thickness of the encapsulation material can be varied by altering the wax:AI stoichiometry. The release kinetics of the encapsulation material can be altered by changing the stoichiometry of glycerin in the formulation.

### Example 12

### Hydrophobic Encapsulation with Wax and a Copolymer Additive

To enhance the stability of AI granules in warmer climates, a small amount of higher melting point olefinic polymer or copolymer can be added to the formulation that encapsulates the AI. For this Example, 0.7 g of wax (Aldrich, m.p 55 C) and 0.3 g of Poly(ethylene-co-vinylacetate) copolymer were dry mixed in a plastic container. To this container, 9 g of the AI metribuzin was added. The container was then loaded into a high shear mixer (FlackTek DAC 150 FVZ-K (FlackTek, Landrum, S.C.)). The mixture was shear mixed at ˜2000 rpm for 10 mins. The high shear melted the wax and formed a coat on the AI granules. The thickness of the encapsulation material can be varied by altering the wax:AI stoichiometry. The thermal stability of formulation can be altered by changing the stoichiometry of the higher melting copolymer in the encapsulation material.

### Example 13

### Chitosan Overcoat of the Encapsulated AI

The hydrophobically encapsulated AI from Examples 4, 9, 10, 11 and 12 can be modified with chitosan, as set forth in Example 2. This chitosan overcoat can allow attachment of the hydrophobically encapsulated AI to substrates such as soil.

### Example 14

### Zein Overcoat of the Encapsulated AI

The hydrophobically encapsulated AI from Examples 4, 9, 10, 11, and 12 can be modified with Zein, as set forth in Example 3. This Zein overcoat can allow attachment of the hydrophobically encapsulated AI to substrates such as soil.

### Example 15

### SMI Overcoat of the Encapsulated AI

100 mg of imidized styrene maleic anhydride (SMA® 1000I) was dissolved in 100 mL acidic water (pH 4) to make a 0.1% solution. 9 g of hydrophobically modified AI from experiments 9, 10 and 12 was suspended in the SMA® 1000I solution and the pH was raised slowly using dilute NaOH to pH 8 to enable precipitation of SMA® 1000I on the AI granules. The same protocol can be applied to AIs modified as set forth in Example 11. This SMA® 1000I overcoat for AIs can allow attachment of the hydrophobically modified AI to substrates such as soil.

### Example 16

### Hydrophobic Encapsulation of AI with Soil-Binding Functionality

1 gm of wax (Aldrich, m.p 55 C) and 0.1 g of 1% SMI in acetone are mixed in a glass container. The solvent is allowed to evaporate while the mixture is mixed, forming a uniform coating of SMI on the wax granules. This modified wax is then mixed with AI in a plastic container. The container is then loaded into a high shear mixer such as the FlackTek DAC 150 FVZ-K (FlackTek, Landrum, S.C.). The mixture is shear mixed at ˜2000 rpm for 10 mins. The high shear melts the wax and forms an encapsulation on the AI granules. The small amount of SMI is then trapped in the encapsulation layer resulting in a cationic functionality that becomes exposed when the coated granules are in an aqueous environment, so that they can attach to anionic substrates such as soil. The thickness of the encapsulation material can be varied by altering the wax:AI stoichiometry.

### Example 17

### Hydrophobic Encapsulation of AI with Cellulosic Derivatives

Solutions of cellulose acetate, cellulose butyrate, cellulose acetate butyrate can be made in acetone at a concentration of 0.1%. The AI to be modified in dry powder form is agitated constantly in a reaction vessel while the cellulose-derivative solution is added slowly. The speed of mixing distributes the cellulosic solution throughout resulting in a uniform coating. The solvent is slowly evaporated leaving behind a stable cellulosic coating on the AI granules.

### Example 18

### Hydrophobic Encapsulation of AI with Cellulosic Derivatives and SMI

To the cellulosic solutions described in example 17 are added a small amount of SMI to enable introduction of cationic groups that have affinity for anionic substrates such as soil. The AI to be modified in dry powder form can be agitated constantly in a reaction vessel while 0.1% cellulose acetate solution with 0.1% by weight of a SMI solution (e.g., SMA® 1000I) solution is added slowly. The speed of mixing distributes the cellulosic solution throughout resulting in a uniform coating of the AI. The solvent is slowly evaporated, leaving behind a stable cellulosic coating on the AI granules with a small amount of SMI that segregates to the surface owing to the low surface energy of the styrene blocks in the SMI. The cationic groups in the SMI have an affinity for the soil that can allow binding of the AI thereto.

### Example 19

### Plasticized Cellulosic Encapsulants

The cellulosic derivative solutions described in Examples 17 and 18 can be modified with a small amount (0.1% by wt) of plasticizers such as glycerin. The combined solution is then added to the AI granules under agitation as in Examples 17 and 18.

### Example 20

### Use of Absorbent Beads to Encapsulate AI

Crosslinked Poly(acrylic acid) beads can be used to imbibe and trap AI molecules for sustained delivery. A 1% solution of AI is made in DI water. The crosslinked absorbent polymer beads are mixed with the AI solution in the ratio of 1:100. The mixture is agitated until all the AI solution was absorbed into the beads and a dry blend of beads was seen. The beads are then dried at 50° C. to remove water and produce polymer beads encapsulating the AI.

### Example 21

### Providing AI-Imbibed Beads with Soil-Binding Polymeric Coating

A 0.1% solution of chitosan can be added to AI imbibed beads made in Example 20. Chitosan to bead ratio is 1:100. The chitosan readily binds to the anionic polymer surface resulting in a robust ionic bonding between the two. The amine groups on chitosan can bind to the soil and attach the AI-imbibed beads thereto.

### EQUIVALENTS

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification and the claims are to be understood as being modified in all instances by the term “about.” Accordingly, unless indicated to the contrary, the numerical parameters set forth herein are approximations that can vary depending upon the desired properties sought to be obtained by the present invention.

While this invention has been particularly shown and described with references to preferred embodiments thereof, it will be understood by those skilled in the art that various changes in form and details may be made therein without departing from the scope of the invention encompassed by the appended claims.

What is claimed is:

1. A formulation comprising:
a polymer and a biologically active ingredient,
wherein the polymer interacts with the biologically active ingredient by pi-pi stacking;
and wherein the biologically active ingredient is an aromatic compound.

2. The formulation of claim 1, wherein the biologically active ingredient is an agricultural active ingredient.

3. The formulation of claim 2, wherein the agricultural active ingredient is selected from the group consisting of an herbicide, an insecticide, and an anti-fungal agent.

4. The formulation of claim 3, wherein the agricultural active ingredient is an herbicide.

5. The formulation of claim 4, wherein the herbicide is water-soluble.

6. The formulation of claim 4, wherein the herbicide is sparingly soluble in water.

7. The formulation of claim 5, wherein the herbicide comprises an agent selected from the group consisting of a phenoxy acid herbicide, a benzoic acid herbicide, a pyridine herbicide, or any combination thereof.

8. The formulation of claim 4, wherein the herbicide comprises a phenoxyacetic herbicide, a phenoxybutyric herbicide, a phenoxypropionic herbicide, an arylaniline herbicide, a chloroacetanilide herbicede, a sulfonamide herbicede, a phenoxy herbicides, or any combination thereof.

9. The formulation of claim 8, wherein the herbicide is selected from the group consisting of 2,4-dichlorophenoxyacetic acid, (4-chloro-2-methylphenoxy) acetic acid, mecoprop, dicamba, dithiopyr, benxyloprop, metalochlor, asulam and bromofenoxim.

10. The formulation of claim 1, wherein the polymer is a styrene maleimide polymer or a styrene maleic anhydride polymer.

11. The formulation of claim 1, wherein the polymer possesses a chemical moiety capable of interacting with a substrate.

12. The formulation of claim 11, wherein the substrate is a non-agricultural substrate.

13. The formulation of claim 11, wherein the substrate is an agricultural substrate.

14. The formulation of claim 13, wherein the substrate is soil or humus.

15. A formulation comprising a biologically active ingredient and a particle, wherein the biologically active ingredient is directly or indirectly attached to a particle and wherein the biologically active ingredient is an aromatic compound that exerts a biological effect on a substrate.

16. The formulation of claim 15, wherein the biologically active ingredient is an agricultural active ingredient.

17. The formulation of claim 16 wherein the agricultural active ingredient is selected from the group consisting of an herbicide, an insecticide, and an anti-fungal agent.

18. The formulation of claim 16, wherein the agricultural active ingredient is an herbicide.

19. The formulation of claim 18, wherein the herbicide is water-soluble.

20. The formulation of claim 18, wherein the herbicide is sparingly soluble in water.

21. The formulation of claim 18, wherein the herbicide comprises an agent selected from the group consisting of a phenoxy acid herbicide, a benzoic acid herbicide and a pyridine herbicide.

22. The formulation of claim 18, wherein the herbicide comprises phenoxyacetic herbicide, phenoxybutyric herbicide, and/or a phenoxypropionic herbicide.

23. The formulation of claim 18, wherein the herbicide is selected from the group consisting of 2,4-dichlorophenoxyacetic acid, (4-chloro-2-methylphenoxy) acetic acid, mecoprop, dicamba, dithiopyr, benxyloprop, metalochlor, asulam and bromofenoxim.

24. The formulation of claim 15, wherein the particle comprises a filler selected from the group consisting of precipitated calcium carbonate, clay, sand, diatomaceous earth, zeolite and silica.

25. The formulation of claim 15, wherein a polymer is attached to the particle and wherein the polymer interacts with the active ingredient.

26. The formulation of claim 25, wherein the polymer interacts with biologically active ingredient by pi-pi stacking.

27. The formulation of claim 25, wherein the polymer is a styrene maleic anhydride polymer.

28. The formulation of claim 27, wherein the surface of the particle is modified with an agent that attaches to the styrene maleic anhydride polymer.

29. The formulation of claim 28, wherein the agent is chitosan.

30. The formulation of claim 26, wherein the polymer is a copolymer of polystyrene and a polymer selected from the group consisting of polyethylene glycol and polypropylene glycol.

31. The formulation of claim 15, comprising more than one biologically active ingredient.

32. The formulation of claim 31, comprising more than one type of polymer.

33. The formulation of claim 15, wherein the particle is a porous particle.

34. A formulation for sustained or controlled release of a biologically active ingredient comprising a core and a coating on the surface of the core, wherein the core comprises the biologically active ingredient and wherein the coating comprises a polymer and wherein the biologically active ingredient is an aromatic compound that exerts a biological effect on a substrate.

35. The formulation of claim 34, wherein the biologically active ingredient is an agricultural active ingredient.

36. The formulation of claim 35, wherein the agricultural active ingredient is selected from the group consisting of an herbicide, an insecticide, an anti-fungal agent, an insecticide and a fertilizer.

37. The formulation of claim 35, wherein the agricultural active ingredient is an herbicide.

38. The formulation of claim 37, wherein the herbicide is water-soluble.

39. The formulation of claim 37, wherein the herbicide is sparingly soluble in water.

40. The formulation of claim 37, wherein the herbicide comprises an agent selected from the group consisting of a triazine, alachlor, benazolin, benzatone, imazapyr, triclopyr and a sulfonyl urea base herbicide.

41. The formulation of claim 34, wherein the polymer is selected from the group consisting of a PPO-PEO copolymer and a copolymer of acrylic monomers.

42. The formulation of claim 34, wherein the polymer is a polycation.

43. The formulation of claim 42, wherein the polycation is selected from the group consisting of a cationic protein, a glycoprotein, imidized styrene maleic anhydride, zein, casein, DADMAC, chitosan, and a polyamine.

44. The formulation of claim 34, wherein the core comprises the biologically active ingredient in a matrix comprising wax, oil, olefin polymer or copolymer, a fatty acid, or a combination of any of thereof.

45. A method of attaching or retaining an agricultural active ingredient on an agricultural substrate comprising:
delivering to a treatment area a formulation comprising the agricultural active ingredient and a polymer, wherein:
the polymer interacts with the active ingredient by pi-pi stacking,
the formulation interacts with or attaches to the substrate,
the agricultural active ingredient is an aromatic compound that exerts a biological effect on a substrate, and
the treatment area comprises the substrate.

46. The method of claim 45, wherein the substrate is soil or humus.

47. The method of claim 45, wherein the substrate is a plant surface.

48. A method of attaching or retaining an agricultural active ingredient on an agricultural substrate comprising:
delivering to a treatment area a formulation comprising the agricultural active ingredient and a particle, wherein:
the agricultural active ingredient is directly or indirectly attached to a particle,
the formulation interact with or attaches to the substrate,
the active ingredient is an aromatic compound exerts a biological effect on a substrate,
and the treatment area comprises the substrate.

49. The method of claim 48, wherein the substrate is soil or humus.

50. The method of claim 48, wherein the substrate is a plant surface.

51. The method of claim 48, wherein the formulation is delivered by spraying.

52. A method of controlling the release of a biologically active ingredient comprising:
delivering to a treatment area a formulation comprising a core and a coating on the surface of the core, wherein:
the core comprises the biologically active ingredient,
the coating comprises a polymer and wherein the biologically active ingredient is an aromatic compound that possesses biological activity against a substrate, and
the treatment area comprises the substrate.
